(19) United States
(12) Patent Application Publication
(10) Pub. No.: US 2023/0072402 A1
(43) Pub. Date: Mar. 9, 2023

(54) PET WASTE DISPOSAL BAGS

(71) Applicant: Avery Nottke, Barrington, IL (US)

(72) Inventor: Carter Paul Nottke, Barrington, IL (US)

(21) Appl. No.: 17/940,228

(22) Filed: Sep. 8, 2022

Related U.S. Application Data
(60) Provisional application No. 63/241,772, filed on Sep. 8, 2021.

(51) Int. Cl. B65F 1/00; E01H 1/12; A61L 2/26; A61L 2/18

(52) U.S. Cl. CPC B65F 1/0006; A61L 2/18; A61L 2/26; E01H 1/1206; A61L 2202/23; B65F 2250/105; E01H 2001/124

(57) ABSTRACT

Pet waste disposal bags and a method of manufacturing pet waste disposal bags are disclosed. The pet waste disposal bag includes a body having an opening configured to provide entry to an interior space sized for at least a hand of the user. The body is biodegradable and includes one or more sanitizing components affixed. Upon sanitation or cleansing of the applicable surface or substance, the disposal bag is configured to be inverted allowing the one or more sanitizing components to be housed within the interior space.

### CROSS REFERENCE

This application claims the benefit of U.S. provisional application number 63/241,772, filed Sept. 8, 2021, the entirety of which is hereby incorporated by reference.

### FIELD OF THE INVENTION

The present invention relates generally to disposable bags, and, more particularly, relates to disposable bags that include a Sanitizing component that are configured for cleanup and removal of pet waste.

### BACKGROUND OF THE INVENTION

Sanitizing components have been used over the past few decades for multiple purposes. Primarily, Sanitizing components are used to sanitize a surface and/or object in a prompt manner and clean up unsanitary matter. Due to the Sanitizing components including one or more anti-bacterial components, such as an antiseptic, Sanitizing components must be packaged and contained in a manner that prevents the applicable anti-bacterial components from drying out resulting in the Sanitizing component becoming less effective.

A major drawback of pet ownership is collecting and disposing of the pet’s fecal matter in public places. For example, most public places are subject to rules or ordinances that requires pet owners pick up after their pet in public places. An inherently unsanitary aspect of collecting and disposing of pet waste is the lack of ability to sanitarily dispose of the pet waste without the owner coming into direct contact with the waste. Currently, pet waste bags provide a means to collect and dispose of pet waste. Typically a pet owner will insert their hand inside the bag, so that the bag extends over their hand and wrist. The pet owner can then grasp the waste, using the bag material as a barrier between the waste matter and their hand, and then, while grasping the waste, with their other hand pull the open end of the bag over their hand and the waste matter, thereby turning the bag inside out with the waste matter now on the inside of the bag. By utilizing biodegradable materials that do not negatively impact the environment, these bags can be disposed of in public waste receptacles, or carried home for disposal. While such bags provide a means to remove the bulk of the waste matter, the surface the waste matter was disposed on, and the pet, may need further sanitary attention. That is, the surface where the waste matter was deposited by the pet may require further cleaning than simply picking up the majority of the waste matter, and the backside of the pet may have become fouled and it may therefore be desirable to clean the pet. Currently, in order for a pet owner to clean the pet in these circumstances, the pet owner has to carry Sanitizing components on them as well; however, not all Sanitizing components are conducive to being used on the bodies of pets, and the pet owner may not wish to expose their skin to the waste matter while cleaning the pet.

Therefore, a need exists for a pet waste disposable bag that circumvents the aforementioned issues.

### SUMMARY OF THE DISCLOSURE

In some embodiments, the disclosure provides a sanitary pet waste disposal bag including an attached Sanitizing component. The sanitary pet waste disposal bag is configured to allow the pet owner to properly collect and dispose of pet waste in a sanitary manner while providing a mechanism that allows the pet owner to clean a surface of the pet as well as the surface area be it indoors or outside in a public place.

In some embodiments, the disclosure provides the method of manufacturing of the sanitary pet waste disposal bag.

In some embodiments, a sanitary pet waste disposal bag includes a mitten body including an attached Sanitizing component in which the mitten body includes an interior space shaped and sized to fit at least one hand of a user and the mitten body is configured to be inverted resulting in housing of the applicable substance in contact with the attached Sanitizing component within the interior space.

In accordance with some embodiments of the inventive disclosure, there is provided a disposable waste bag that includes a biodegradable planar body having a first end and a second end and sides defined between the first and second ends. The first end includes an opening, and the second end and sides are sealed to define an interior of the biodegradable planar body. The bag further includes a sanitizing component affixed to at least one of the first or second ends on an outside surface of the biodegradable planar body.

In accordance with a further feature, the sanitizing component is affixed adjacent the second end and spans a width of the biodegradable planar body.

In accordance with a further feature, wherein the sanitizing component is affixed adjacent the opening.

In accordance with a further feature, there is further included a removable flap that is affixed to the outside of the biodegradable planar body over the sanitizing component.

In accordance with a further feature, the sanitizing component is a first sanitizing component, the disposable waste bag further comprising a second sanitizing component affixed to the outside surface of the biodegradable planar body.

In accordance with a further feature, the sanitizing component is removably affixed to the outside surface.

In accordance with a further feature, there is further included a divider inside the body that divides the interior space into separate two portions.

In accordance with some embodiments of the inventive disclosure, there is provided a disposable waste bag that includes a body having a first end and a second end. The body is made of a sheet of liquid impermeable material surrounding an interior space that is open at the first end and otherwise sealed. The bag also includes a sanitizing component affixed to an outside surface of the body. The sanitizing component is made of a fibrous or porous material that holds a sanitizing fluid.

In accordance with a further feature, the sanitizing component is affixed to the outside of the body adjacent the first end of the body.

In accordance with a further feature, the sanitizing component is affixed to the outside of the

In accordance with some embodiments of the inventive disclosure, there is provided a pet waste disposal article that includes a body comprised of a sheet material in the form of a bag having an opening, an interior, and an exterior, the opening being configured to allow a user to place a hand into the interior, and the sheet material being liquid impermeable. The pet waste disposal bag further includes a sanitizing component affixed on the exterior of the body. The sanitizing component is made of a material that holds a sanitizing fluid and is configured to wet a surface to be sanitized by the sanitizing component with the sanitizing fluid upon the sanitizing component being placed into contact with the surface.

In accordance with a further feature, the sanitizing component is permanently attached to the body.

Although the invention is illustrated and described herein as embodied in a pet waste disposal bag, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

"In the description of the embodiments of the present invention, unless otherwise specified, azimuth or positional relationships indicated by terms such as "up", "down", "left", "right", "inside", "outside", "front", "back", "head", "tail" and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

In the description of the embodiments of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the article being referenced. Those skilled in the art can understand the specific meanings of the above-mentioned terms in the embodiments of the present invention according to the specific circumstances.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a prospective view of a first pet waste disposal bag, according to an exemplary embodiment;

FIGS. 2A-2D show a use example of a pet waste disposal bag;

FIG. 3 is a prospective view of a second pet waste disposal bag, according to an exemplary embodiment;

FIG. 4 is a side view of a third pet waste disposal bag, according to an exemplary embodiment;

FIG. 5A is a side view of the third pet waste disposal bag of FIG. 4 including a Sanitizing components component, according to an exemplary embodiment;

FIGS. 5B-5C shown side views of a disposable pet waste bag having a removable flap covering a sanitizing component.

FIG. 6A is a side view of a fourth pet waste disposal bag, according to an exemplary embodiment;

FIGS. 6B and 6C show side and end views, respectively, of a disposable pet waste bag in which the internal space is divided into separate portions for each of the user’s hands.

FIGS. 7A-7C show a packaging arrangement for pet waste disposal bags, in accordance with some embodiments; and

FIG. 8 shows an example of a disposable pet waste bag in which the sanitizing component is removably affixed to the external surface of the bag, in accordance with some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

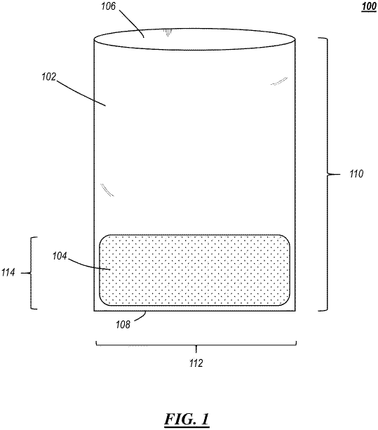

FIG. 1 shows a first pet waste disposal bag 100, according to an exemplary embodiment. In some embodiments, pet waste disposal bag 100, in a folded state, includes a generally planar body 102 in a rectangular configuration that is sealed along three of the four edges and open at the top 106. A user can insert things into the bag 100 by placing it though the opening 106 where the thing will then be surrounded by the body 102 of the bag. The body 102 is made of a liquid impermeable material, such as, for example, plastic. A sanitizing component 104 is provided on the outside of the bag closer to the bottom 108 of the bag 102. The sanitizing component can be a woven material that is affixed to the outside surface of the body 102 and which holds a sanitizing fluid. The sanitizing component 104 can alternatively be a non-woven fibrous material, or a porous material (e.g. foam or sponge) that can hold and retain a sanitizing fluid, and will expel, emit, or otherwise apply the sanitizing fluid onto a surface upon the sanitizing component making contact with a surface to be sanitized, or any surface in general upon sufficient contact being made. The sanitizing fluid is held in the sanitizing component 104 by wicking action among the fibers or pores of the sanitary component 104. Upon pressing the sanitizing component 104 against something, some of the sanitizing fluid is transferred to that thing by the pressure exerted on the sanitizing component 104 at the contact point or points. The sanitizing fluid can include forms of alcohol-based or non-alcohol germicidal fluids. The sanitizing component 104 can be affixed to the body 102 by an adhesive, or by heat staking it to the material of the body 102 where the material of the body 102 and the material of the sanitizing component 104 arejoined by heating them while in contact with each other.

While it will be appreciated that various shapes, dimensions, and sizes of bag 100 and sanitizing component 104 are within the spirit and scope of the disclosure. In a preferred embodiment, the length 110 of body 102 can be approximately 8-9 inches, the width 114 of sanitizing component 104 is about 3-4 inches, and the width 112 of body is approximately 5-6 inches. In some embodiments, bag 100 includes at least one opening 106 configured to support placement of at least one of the user’s hands inside the body 102. The placement of the hand through the opening 106 allows the user to grab the pet’s waste without the hand coming into direct contact with the waste. In some embodiments, the hand can be removed from the opening while the user’s hand is in contact with the waste by using their other hand to pull sanitizing component 104 upward. Turning the bag 100 so that the sanitizing component 104 is oriented upward allows the sanitizing component 104 to be upwardly exposed while the pet waste is retained on the exterior of bag 100 opposite the sanitizing component 104. The pet owner may utilize either upwardly or downwardly exposed sanitizing component 104 to clean the pet, or clean the pet first, and then pick up the waste. In addition, the user can use the bag 100 to pick up waste on the opposite si

It is to be understood that although sanitizing component 104 may include one or more anti-microbial fluids selected to destroy germs, harmful bacteria, viruses, yeast, mold, fungi, mycoplasma, protozoa, or other microorganisms, the distribution of anti-microbial components may be adjusted in order to allow sanitizing component 104 to be safely used on pets for the purpose of cleansing or improving odor of the pet. The aforementioned may also be used with a harsher disinfectant to allow for sterilizing surfaces in a hospital setting.

It is to be understood that pet waste disposal bag 100 and any other embodiment disclosed herein are not limited to pet waste disposal and pet sanitation; in some embodiments, the pet waste disposal bags may be utilized in medical environments (hospitals, doctors' offices, etc.), athletic environments (gyms, locker rooms, etc.), camping environments, and any other applicable environments where sanitation is necessary.

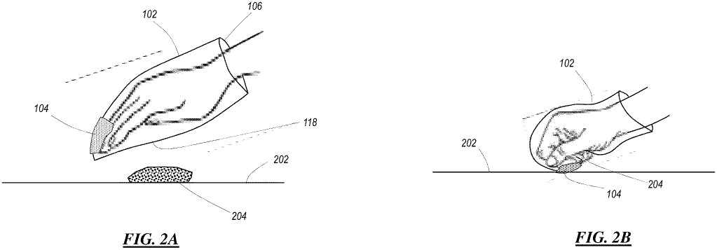

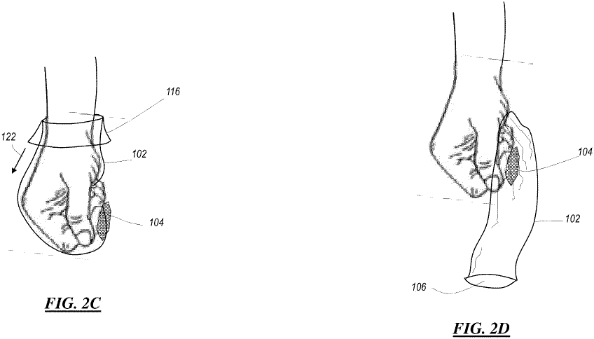

FIGS. 2A-2D show a use example of a pet waste disposal bag using pet waste disposal bag 100 as an example. In FIG. 2A a user has inserted their hand into the inside of the body 102. As a result, the sanitizing component 104, located near the end of the body 102 opposite the opening 106, is positioned over the user’s fingertips. The opposite side 118 is facing down. An amount of pet waste 204 sits on a surface 202, such as a floor, sidewalk, or other such surface. The user can them move their hand downward so that the waste 204 comes into contact with opposite side 118 (at the outside) of the bag under the user’s hand, wherein, using a grasping motion with the users fingers, the pet waste 204 can be picked up, as shown in FIG. 2B. The curling of the fingers in making the grasping motion brings the sanitizing component 104 to the lowermost position, over the area where the pet waste 204 was located, allowing the user to press the sanitizing component 104 onto the surface 202 to apply the sanitizing fluid to the surface 202 and further clean up any residue. That is, the sanitizing component 104 wets the surface 202 (or any other surface) upon the sanitizing component 104 being pressed into contact with the surface 202. Once the pet waste 204 is removed from the surface 202 and the surface 202 sanitized, then in FIG. 2C the user can, using their other hand, turn the body 102 inside out by pulling the open end of the body 102 over the body 102, as indicated by arrow 122. Once the bag body 102 has been fully inverted, as in FIG. 2D, the user can relax their hand as the pet waste will be trapped inside the now inverted bag body 102, and the user can tie off the open end and dispose of the bag and waste appropriately.

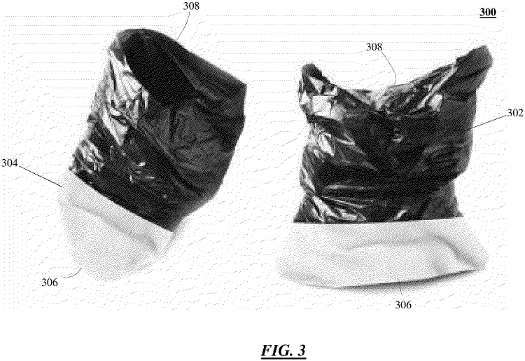

FIG. 3 shows a pair of pet waste disposal bags 300, according to an exemplary embodiment. In some embodiments, bags 300 may each include a receptacle portion 302 and a sanitizing component 304. In some embodiments, at least one of receptacle portion 302 or sanitizing component 304 includes an opening 306 configured to support placement of a hand of the pet owner inside the bag 300. In some embodiments, bag 300 may be configured to be worn by the pet owner much like a mitten in which bag 300 is removed from hand by pulling sanitizing component 304 toward the opposite end 308. The user can place their hand inside the bag, grasp the pet waste through the bag at the opposite end 308, hold the pet waste while using the sanitizing component 304 to clean the pet or the surface from which the waste was removed, and then pull the end of the bag where the sanitizing component 304 is located such that the opening 306 passes along the bag 300 and the bag 300 becomes turned inside out. Thereafter the bag 300 can be secured closed such as be tying the bag, and the bag 300 including the pet waste and soiled sanitizing component can be disposed of.

In some embodiments, the exterior of bag 300 is composed of biodegradable material while the opening 306, interior surface of bag 300, and sanitizing component 304 may be composed of a fibrous material. In some embodiments, the fibrous material may include natural and/or synthetic materials including but not limited to wool, silk, jute, hemp, cotton, linen, sisal, ramie, rayon, cellulose, ester, polyvinyl derivatives, polyolefins, polyamides, or polyesters.

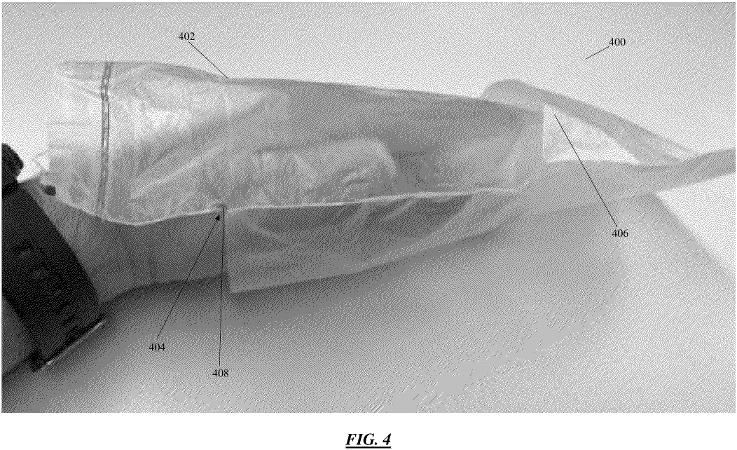

FIG. 4 shows a pet waste disposal bag 400, according to an exemplary embodiment. In some embodiments, pet waste disposal bag 400 includes a mitten body 402 including an open end 404 and a flap 406. In some embodiments, open end 404 opens to an interior space 408 that can be shaped and sized to fit at least one hand of a user. In some embodiments, interior space 408 includes a thumb portion and a finger portion configured to allow the fingers and thumb of the user to be housed in separate sections of interior space 408. In some embodiments, interior space 408 may be shaped and sized to allow the user to wear a glove or other protective mechanism within interior space 408. In some embodiments, flap 406 can be attached with one or more attachment mechanisms such as but not limited to an adhesive, plastic strips, layers, glue, or any other mechanism configured to support attachment and/or retention of sanitizing components. In some embodiments, flap 406 may be a detachable member configured to be removable from mitten body 402. The detachable member may be a mechanism to prevent exposure, contamination, or drying out of the one or more sanitizing components configured to be affixed to mitten body 402. In some embodiments, flap 406 may be allocated on any applicable portion of mitten body 402.

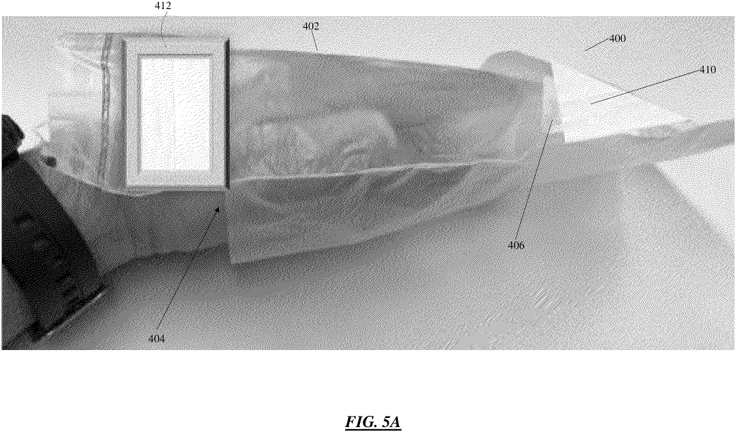

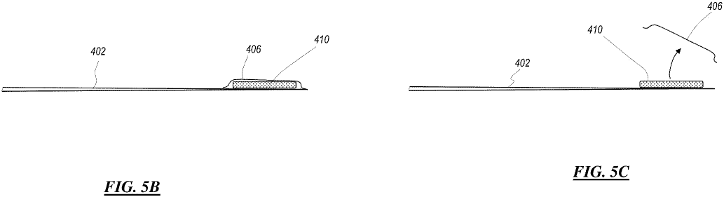

Referring now to FIG. 5A, in another embodiment of pet waste disposal bag 400, the bag 400 includes a sanitizing component 410. In some embodiments, sanitizing component 410 is attached at or proximate to flap 406, or covered by the flap 406. Sanitizing component 410 may be affixed to mitten body 402 at any applicable location that allows sanitizing component 410 to come into contact with the surface and/or applicable substance that needs to be sanitized, cleaned, or sustained without coming into direct contact with one or more hands of the user. Sanitizing component 410 may be composed of a biodegradable material and may include one or more chemical solutions and/or cleansing/disinfecting agents such as antimicrobial compounds. Sanitizing component 410 may be a Cavicide wipe, a chlorhexidine wipe, Clorox wipe, or any other applicable antimicrobial/cleansing agent based wipe.

In some embodiments, the chemical solutions and cleansing agents within the sanitizing component 410 may be protected or concealed by a detachable cover from outside exposure that may dilute or contaminate sanitizing component 410. It is to be understood that the manner in which sanitizing component 410 is affixed (and concealed) to mitten body 402 is intended not to reduce the biodegradability of pet waste disposal bag 400. For example, plastic strips may be used to either glue, seal, or sew on to mitten body 402; however, the plastic strips are configured to be peeled or removed from mitten body 402.

In FIGS. 5B-5C an embodiment where the sanitizing component 410 is located under the flap 406. The body is shown from the side, flattened, with the sanitizing component 410 under the flap 406, which, in this example, is removable. Thus in FIG. 5C, the flap 406 is pulled off, exposing the sanitizing component 410 for use. In this example, the flap protects the sanitizing component from drying out before it is used.

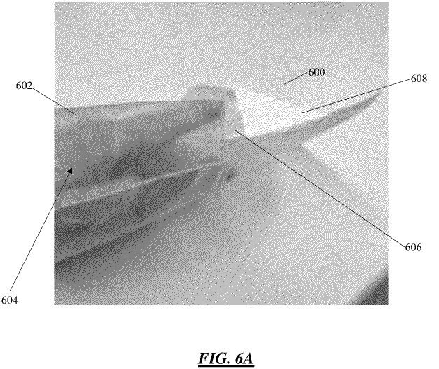

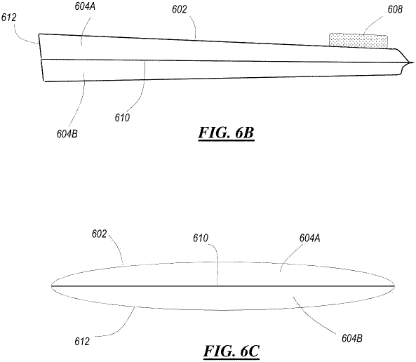

Referring now to FIG. 6A, a pet waste disposal bag 600, according to an exemplary embodiment. In some embodiments, pet waste disposal bag 600 includes a body 602 including an interior space 604 allocated proximate to at least one end of body 602. It is to be understood that interior space 604 is designed and configured to house both hands of the user allowing a dual handed configuration to sanitize and/or clean. In some embodiments, body 602 includes a flap 606 in which flap 606 may be configured to include at least one partition allowing the hands of the user to be housed in separate sections of interior space 604. In some embodiments, flap 606 may include a sanitizing component 608 affixed to an exterior surface of flap 606. It is to be understood that the dual handed configuration allows both palms to face each other while the fingers of the opposing hands are housed in separate sections respectively. In some embodiments, sanitizing component 608 is allocated to a top side and a bottom side of flap 606 allowing the separation of the opposing fingers to create a space designed and configured to come into contact with the surface or substance requiring sanitation or cleansing. It is to be understood that upon the sanitation or cleansing of the applicable surface or substance, body 602 is configured to be inverted allowing sanitizing component 508 to be housed within interior space 604 (which is the former exterior of body 602). In some embodiments, interior space 604 includes a one or more sanitizing components in order to allow the user to effectively sanitize/clean their hands after pet waste disposal bag 60 has come into contact with the applicable surface or substance.

FIGS. 6B and 6C show side and end views, respectively, of the body 602. The body 602 is open at a first end 612, and the interior space 604 is divided by a divider 610 which runs the length of the body from the first end 612 to the opposite end adjacent the sanitizing component 608. The divider 610 divides the interior space 604 into separate portions 604A and 604B, and the user can insert each hand into a different one of the two spaces 604A-B.

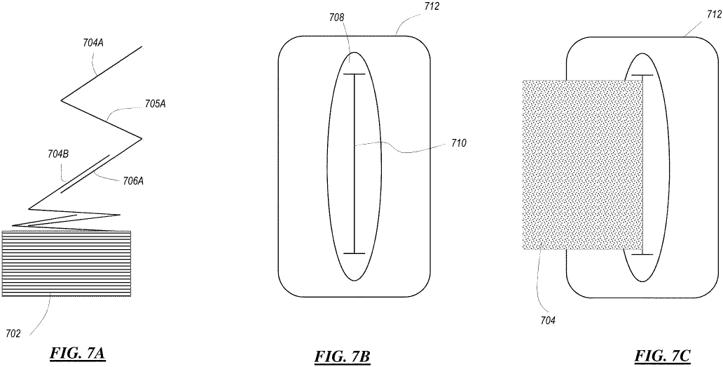

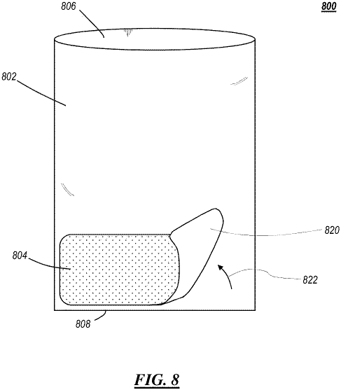

FIGS. 7A-7C show a packaging arrangement for packaging pet waste disposal bags. In FIG. 7A, there is a stack 702 of pet waste disposal bags that are tri-folded on top of each other. That is, each bag is folded into three sections including a upper section 704, middle section 705 and lower section 706. The terms “upper” and “lower” are in relation to the vertical direction of the stack 702. The lower section 706 is disposed under the upper section of the next lower bag. Thus, lower section 706A is positioned under upper section 704B of the next bag. The bags in the stack can be substantially similar to any of the bags described in the foregoing embodiments, or any embodiment consistent with the disclosure. Thus, when the uppermost bag is lifted, the lower section 706A lifts the upper section 704B. When the stack is placed in a dispensing package 712, the folding relationship in the stack allows one bag to be pulled out of the dispensing package 712 such that the upper portion 704 of the next bag is placed in position to be pulled out next, which partially pulls out the next bag, and so on. The dispensing package 712 is generally a vapor impermeable package in which the stack 702 is placed, and the packaging 712 is sealed. An opening section 708 includes a dispensing opening 710 that is configured to substantially seal closed when there is no bag section passing through the opening 710. The sides of the opening 710 can be moved apart to allow the user to grasp the top portion 704 of the next bag in the stack (e.g. the uppermost bag). As shown in FIG. 7C, the top section 704 of a bag being dispensed is drawn through the opening 710 and pulled out, whereupon the opening 710 can self close to seal in moisture and prevent the sanitizing components from drying out.

FIG. 8 shows an alternative pet waste disposal bag 800 in which the sanitizing component 804 is configured to be removed by the user, if the user so desires. The bag 800 includes a body 802 that has an open end 806 at a first end. The second end 808 and the sides between the ends are sealed together to define an interior space. The sanitizing component 804 is removably affixed to the outside surface of the body 802, and can be peeled off by lifting a portion 820, as indicated by arrow 822. Then the user can use the sanitizing component separately from the bag. As an alternative to a stack 702, the bags can also be provided in a roll when to top portion 704 of each successive bag in the roll overlaps the lower portion 706 of the preceding bag.

In some embodiments, a method of manufacturing for a pet waste disposal bag is provided. In some embodiments, the method includes the sanitizing component being integrated to the pet waste disposal bag via heat sealing or any other applicable mechanism for affixing. In some embodiments, the method of manufacturing the disposal bag may be accomplished by robotics, assembly line structure, or any other applicable manufacturing process known to those of ordinary skill in the art. In some embodiments, the method of manufacturing for the pet waste disposal bag includes affixing a treated textile material including one or more antimicrobial agents including but not limited to penicillin G, procaine penicillin, benzathine penicillin, and penicillin V, or any other applicable antimicrobial agents and/or microorganism killing agent. Heat, glue/adhesive, and other mechanisms to securely affix the textile material to the pet waste disposal bag is within the scope and spirit of the disclosure.

The disclosed embodiments solve the problem of having to carry both bags and sanitizing wipes in order to clean up surfaces or parts of a pet when the pet defecates. By affixing a sanitizing component to each bag, the user/pet owner can carry just several bags without having to also carry a separate container for sanitizing wipes. The user can clean using the sanitizing component after picking up waste, or the user can clean up the pet prior to picking up waste where the user’s hand is fully protected by the material of the bag. Optionally, in some embodiments, the user can remove the sanitizing component, holding it with their other hand to clean, and then deposit the used sanitizing component into the inverted bag for disposal.

What is claimed is:

1. A disposable waste bag comprising:
a biodegradable planar body having a first end and a second end and sides defined between the first and second ends;
the first end includes an opening, and the second end and sides are sealed to define an interior of the biodegradable planar body; and
a sanitizing component affixed to at least one of the first or second ends on an outside surface of the biodegradable planar body.

2. The disposable waste bag of claim 1, wherein the sanitizing component is affixed adjacent the second end and spans a width of the biodegradable planar body.

3. The disposable waste bag of claim 1, wherein the sanitizing component is affixed adjacent the opening.

4. The disposable waste bag of claim 1, further comprising a removable flap that is affixed to the outside of the biodegradable planar body over the sanitizing component.

5. The disposable waste bag of claim 1, wherein the sanitizing component is a first sanitizing component, the disposable waste bag further comprising a second sanitizing component affixed to the outside surface of the biodegradable planar body.

6. The disposable waste bag of claim 1, wherein the sanitizing component is removably affixed to the outside surface.

7. The disposable waste bag of claim 1, further comprising a divider inside the body that divides the interior space into separate two portions.

8. A disposable waste bag comprising:
a body having a first end and a second end, the body comprised of a sheet of liquid impermeable material surrounding an interior space that is open at the first end and otherwise sealed; and
a sanitizing component affixed to an outside surface of the body, the sanitizing component comprised of a fibrous material that holds a sanitizing fluid.

9. The disposable waste bag of claim 8, wherein the sanitizing component is affixed to the outside of the body adjacent the first end of the body.

10. The disposable waste bag of claim 8, wherein the sanitizing component is affixed to the outside of the body adjacent the second end of the body.

11. The disposable waste bag of claim 8, further comprising a removable flap that covers the sanitizing component.

12. The disposable waste bag of claim 8, further comprising a divider inside the body that divides the interior space into separate two portions.

13. The disposable waste bag of claim 8, wherein the sanitizing component is removably affixed to the outside surface.

14. The disposable waste bag of claim 8, wherein the sanitizing component is a first sanitizing component, the disposable waste bag further comprising a second sanitizing component affixed to the outside surface of the biodegradable planar body.

15. A pet waste disposal article, comprising:
a body comprised of a sheet material in the form of a bag having an opening, an interior, and an exterior, the opening configured to allow a user to place a hand into the interior, the sheet material being liquid impermeable; and
a sanitizing component affixed on the exterior of the body, the sanitizing component comprised of a material that holds a sanitizing fluid and which is configured to wet a surface to be sanitized by the sanitizing component with the sanitizing fluid upon the sanitizing component being placed into contact with the surface.

16. The pet waste disposal article of claim 15, wherein the sanitizing component is permanently attached to the body.

17. The pet waste disposal article of claim 15, wherein the sanitizing component is affixed to the outside of the body adjacent the first end of the body.

18. The pet waste disposal article of claim 15, wherein the sanitizing component is affixed to the outside of the body adjacent the second end of the body.

19. The pet waste disposal article of claim 15, further comprising a removable flap that covers the sanitizing component.

20. The pet waste disposal article of claim 15, further comprising a divider inside the body that divides the interior space into separate two portions.
